(19) United States
(12) Patent Application Publication
(10) Pub. No.: US 2010/0215730 A1
(43) Pub. Date: Aug. 26, 2010

(54) Compositions for Nasal Administration of Phenothiazines

(75) Inventors: David GUY, Rancho Santa Fe, CA (US); Cam GALLAGHER, San Diego, CA (US); David HALE, Encinitas, CA (US)

Correspondence Address: WILSON, SONSINI, GOODRICH & ROSATI, 650 PAGE MILL ROAD, PALO ALTO, CA, 94304-1050, US

(73) Assignee: INTREPID THERAPEUTICS, INC., Encinitas, CA (US)

(21) Appl. No.: 12/709,128

(22) Filed: Feb. 19, 2010

Related U.S. Application Data
(60) Provisional application No. 61/154,326, filed on Feb. 20, 2009.

(51) Int. Cl. A61K 31/5415; A61K 9/127; A61P 35/00

(52) U.S. Cl. 424/450; 514/224.8

(57) ABSTRACT

Provided herein are pharmaceutical compositions comprising phenothiazines or derivatives thereof that are formulated for nasal administration. Also provided herein are methods of utilizing the same.

### CROSS-REFERENCE

This application claims the benefit of U.S. Provisional Application No. 61/154,326 filed Feb. 20, 2009 which is incorporated herein by reference in its entirety.

### BACKGROUND OF THE INVENTION

Phenothiazines constitute a broad class of drugs that have antipsychotic properties, although they may also cause severe side effects such as drowsiness, dizziness, blurred vision, abnormal menstrual flow, rash, low blood pressure, and/or distorted voluntary movements of the body (dystonic reaction). In some instances, cholestatic jaundice or fever with grippe-like symptoms may occur. Some phenothiazines, such as prochlorperazine also have anti-emetic properties and are used in the treatment of vertigo. Prochlorperazine is conventionally administered via the oral, rectal, intravenous or intramuscular routes. Following intramuscular administration of prochlorperazine edisylate, the drug has an onset of action within ten to twenty minutes and a duration of action of three to four hours. The systemic administration of conventional dosage forms of prochlorperazine is associated with variable blood levels of the drug in patients. Described herein are pharmaceutical compositions for the nasal and/or sublingual administration of phenothiazines, particularly prochlorperazine, for the treatment of nausea and emesis. Intranasal and/or sublingual administration of the compositions described herein avoids the disadvantages associated with other routes of administration and provides rapid relief from nausea or emesis.

### SUMMARY OF THE INVENTION

Described herein are pharmaceutical compositions for increasing the absorption of phenothiazines via the nasal or inhalation route in a subject. The compositions provided herein prevent, lessen, reverse or treat nausea or emesis in a subject. Further, the compositions described herein allow for improved delivery of the compound into the circulatory system of a subject and provide rapid relief from symptoms of nausea or emesis.

Provided herein, in certain embodiments, are pharmaceutical compositions comprising a phenothiazine or phenothiazine derivative, or a salt thereof, or any combination thereof, and at least one permeation enhancer, wherein the pharmaceutical composition is formulated for nasal administration.

In some embodiments, the phenothiazine is prochlorperazine (2-chloro-10-[3-(4-methylpiperazin-1-yl)propyl]phenothiazine) a prochlorperazine derivative, or a salt thereof, or any combination thereof.

In some embodiments, the permeation enhancer reduces the concentration of the phenothiazine in the composition necessary to exert a therapeutic effect in a subject compared to a composition without the permeation enhancer.

In some embodiments, the compositions further comprise an alkyl glycoside having a hydrophobic alkyl group joined by a linkage to a hydrophilic saccharide.

In some embodiments, the permeation enhancer is selected from: (a) a surfactant; (b) a bile salt; (c) a phospholipid additive, (d) a mixed micelle (e) a liposome; (f) an alcohol; (g) an enamine; (h) an NO donor compound; (i) a long-chain amphipathic molecule; (j) a small hydrophobic penetration enhancer; (k) sodium or a salicylic acid derivative; (l) a glycerol ester of acetoacetic acid; (m) a cyclodextrin or beta-cyclodextrin derivative; (n) a medium-chain fatty acid; (o) a chelating agent; (p) an amino acid or salt thereof; (q) an N-acetylamino acid or salt thereof; (r) an enzyme degradative to a selected membrane component; and (s) an alkyl glycoside.

In some embodiments, the alkyl glycoside is selected from dodecyl maltoside, tridecyl maltoside, tetradecyl maltoside, sucrose mono-dodecanoate, sucrose mono-tridecanoate, and sucrose mono-tetradecanoate.

In some embodiments, the compositions described herein further comprise a delivery enhancing agent.

In some embodiments, the delivery enhancing agent is selected from a charge-modifying agent, a pH control agent, a degradative enzyme inhibitory agent, a mucolytic or mucus clearing agent, a chitosan, and a ciliostatic agent.

In some embodiments, the pH of the composition is between about 5 and about 8.

Also provided herein is a method of treating nausea or emesis in a subject comprising intranasally administering a composition described herein. In some of such embodiments, the subject is undergoing treatment for cancer (e.g., chemotherapy). In some of such embodiments, the composition comprises prochlorperazine, or a derivative, or a salt, or any combination thereof. Also provided herein is a method of treating nausea or emesis in a subject comprising sublingually administering a composition described herein.

Also provided herein is the use of a composition described herein (e.g., a composition comprising prochlorperazine) for the manufacture of a nasally administered medicament for the treatment of nausea or emesis.

Also provided herein is a method of making a pharmaceutical composition for nasal delivery comprising formulating at least one phenothiazine or phenothiazine derivative, or a salt thereof, or any combination thereof, at least one permeation enhancer, and a pharmaceutically acceptable carrier or excipient, wherein said pharmaceutical composition is suitable for nasal administration.

### INCORPORATION BY REFERENCE

All publications, patents, and patent applications mentioned in this specification are herein incorporated by reference to the same extent as if each individual publication, patent or patent application was specifically and individually indicated to be incorporated by reference.

### DETAILED DESCRIPTION OF THE INVENTION

Provided herein are pharmaceutical compositions for delivery of a phenothiazine or phenothiazine derivative, or a salt thereof, or any combination thereof, via nasal administration to a subject in need thereof. Systemic routes, e.g., oral routes, create a delayed onset of therapeutic relief in a subject in need thereof. Intravenous and/or intramuscular routes of administration create discomfort on administration and may require a hospital visit. Intranasal administration of the pharmaceutical compositions described herein provides safe and rapid therapy to a subject experiencing or suspected of having nausea and/or emesis, with minimal side effects and minimal discomfort to the subject upon administration.

In some embodiments, compositions described herein comprise penetration enhancers and/or delivery enhancers that improve delivery across nasal barriers into the circulatory system of a subject in need thereof. The pharmaceutical compositions described herein allow for the administration of phenothiazines at lower concentrations and reduced frequency of administration to achieve a desired therapeutic effect and thus overcome the attendant side effects of systemic delivery of phenothiazines at high concentrations.

Also provided herein are pharmaceutical compositions for delivery of a phenothiazine or a derivative thereof via sublingual administration to a subject in need thereof. Sublingual delivery allows a phenothiazine to dissolve in the immediate vicinity of the site of administration, allowing the drug to enter directly into the blood stream and exert its pharmacological effect rapidly. Sublingual administration by-passes gastric juices, acid environment and enzymes present in the gastrointestinal tract and the liver which is the target organ for metabolism of a drug that is administered orally. The highly vascular mucosal lining under the tongue where sublingual tablets are placed allows for the delivery of phenothiazines directly into the circulatory system and thus overcomes the attendant side effects associated with administration of phenothiazines via oral administration. Moreover, sublingual administration allows for administration of phenothiazines at lower doses and reduced frequency of administration to achieve a desired therapeutic effect.

### CERTAIN DEFINITIONS

As used herein, the term “subject” is used to mean an animal, preferably a mammal, including a human or non-human. The terms patient and subject may be used interchangeably.

As used herein, amelioration or lessening of the symptoms of a particular symptom, disorder or condition by administration of a particular compound or pharmaceutical composition refers to any decrease of severity, delay in onset, slowing of progression, or shortening of duration, whether permanent or temporary, lasting or transient that is attributed to or associated with administration of the compound or composition.

The term “inhibiting” includes preventing, slowing, or reversing the development of a condition, for example, nausea associated with cancer therapy, or advancement of a condition in a patient necessitating treatment.

Drug absorption” or “absorption” refers to the process of movement of the phenothiazine from the localized site of administration, such as by way of example only, nasal passage, and across a barrier (e.g., the nasal mucosa) into the circulatory system.

The terms “effective amount” or “therapeutically effective amount,” as used herein, refer to a sufficient amount of the phenothiazines being administered that would be expected to relieve to some extent one or more of the symptoms of the disease or condition being treated. For example, an “effective amount” for therapeutic uses is the amount of the phenothiazine(s) including a composition as disclosed herein required to provide a decrease or amelioration in disease symptoms without undue adverse side effects. The term “therapeutically effective amount” includes, for example, a prophylactically effective amount. An “effective amount” of a composition disclosed herein is an amount effective to achieve a desired pharmacologic effect or therapeutic improvement without undue adverse side effects. It is understood that “an effective amount” or “a therapeutically effective amount” varies, in some embodiments, from subject to subject, due to variation in metabolism of the compound administered, age, weight, general condition of the subject, the condition being treated, the severity of the condition being treated, and the judgment of the prescribing physician.

The terms “treat,” “treating” or “treatment,” as used herein, include: reversing, alleviating, abating or ameliorating a disease or condition, for example nausea, or emesis, as well as symptoms of the disease or condition; preventing additional symptoms; ameliorating or preventing the underlying metabolic causes of symptoms; inhibiting the disease or condition, e.g., arresting the development of the disease or condition; relieving the disease or condition; causing regression of the disease or condition; relieving a condition caused by the disease or condition; or stopping the symptoms of the disease or condition either prophylactically and/or therapeutically.

A “prodrug” refers to a compound or agent that is converted into the parent drug in vivo. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. In one embodiment, the prodrug is designed to alter the absorption and/or the transport characteristics of a drug, or to alter other characteristics or properties of a drug. Phenothiazines described herein, in some embodiments, are derivatized into suitable prodrugs.

The term “pharmaceutically acceptable derivatives” of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. In addition, a single-isomer formulation of a racemic compound is also a “pharmaceutically acceptable derivative.”

Pharmaceutically acceptable salts include, but are not limited to, amine salts, including but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. The pharmaceutically acceptable salts also include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. The pharmaceutically acceptable salts of the compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

The term “permeation enhancer” with respect to a composition or ingredient, as used herein, refers to the property of reducing barrier (e.g., nasal membrane) resistance. As used herein, a “permeation enhancer” functions to facilitate the passage of an active substance through a barrier, e.g., a nasal membrane, to protect an active substance from being damaged, degraded or altered as it passes through a barrier, and/or to carry an active substance to a desired target in the body after the active substances passes through a barrier. Permeation enhancers include agents which enhance the release or solubility (e.g., from a formulation delivery vehicle), diffusion rate, penetration capacity and timing, uptake, residence time, stability, effective half-life, peak or sustained concentration levels, clearance and other desired mucosal delivery characteristics (e.g., as measured at the site of delivery, or at a selected target site of activity such as the bloodstream or central nervous system) of a compound(s) (e.g., a phenothiazine). Enhancement of permeation can occur by any of a variety of mechanisms, including, for example, by increasing the diffusion, transport, persistence or stability of the compound, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junction physiology, reducing the viscosity of mucus overlying the mucosal epithelium, reducing mucociliary clearance rates, and other mechanisms.

The terms “enhance” or “enhancing” refer to an increase in or prolongation of absorption (e.g., nasal absorption) of a phenothiazine that is consequent upon administration of the compositions described herein.

The term “delivery enhancing agent” with respect to a composition or ingredient, as used herein, refers to the property of increasing the amount of a therapeutic agent that is delivered into the circulatory system and/or at a selected target site. Enhanced delivery can occur by any variety of mechanisms including, for example, modulating the activity of phenothiazine degradation enzymes, removal of mucus from the nasal membrane and other mechanisms.

The term “phenothiazine” refers to a heterocyclic structure comprising a central 1,4-thiazine six-membered ring with two additional six-membered aromatic carbon rings symmetrically joined at the 1,3- and 5,6-positions.

The term “phenothiazine derivative” includes, prodrugs (e.g., lipid conjugates, esters), pharmaceutically acceptable salts, solvates, isomers, tautomers, metabolites, analogs, or combinations thereof. In some embodiments, compounds described herein are prepared as prodrugs. A “prodrug” refers to an agent that is converted into the parent drug in vivo. Prodrugs are often useful because, in some situations, they are easier to administer than the parent drug. In some other embodiments, the prodrug is bioavailable whereas the parent is not. In other embodiments, the prodrug also has improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the active entity, once inside the cell where water-solubility is beneficial. In other embodiments, the prodrug is a short peptide (e.g., polyaminoacid) bonded to an acid group where the peptide is metabolized to reveal the active moiety. In certain embodiments, the prodrug is a lipid conjugate that aids transport across a biological membrane and is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

To produce a phenothiazine derivative, a pharmaceutically active compound is modified such that the active compound will be regenerated upon in vivo administration. In other embodiments, the derivative is designed to alter the metabolic stability or the transport characteristics of a drug, to mask side effects or toxicity, to improve the flavor of a drug or to alter other characteristics or properties of a drug. In some embodiments phenothiazine derivatives are prodrugs that are designed as reversible drug derivatives, for use as modifiers to enhance drug transport to site-specific tissues. In some embodiments, the design of a prodrug increases the effective water solubility.

In some embodiments, certain sites (e.g. aromatic rings) on the compounds described herein are susceptible to various metabolic reactions, therefore incorporation of appropriate substituents, such as, by way of example only, halogens can reduce, minimize or eliminate this metabolic pathway.

### Intranasal Formulations

Provided herein are intranasal compositions comprising phenothiazines or phenothiazine derivatives. In some embodiments, the compositions comprise one or more permeation enhancers, and/or one or more alkyl glycosides having a hydrophobic alkyl group joined by a linkage to a hydrophilic saccharide.

In some embodiments, the compositions comprise one or more permeation enhancers selected from: (a) a surfactant; (b) a bile salt; (ii) a phospholipid additive, (d) a mixed micelle (e) a liposome; (f) an alcohol; (g) an enamine; (h) an NO donor compound; (i) a long-chain amphipathic molecule; (j) a small hydrophobic penetration enhancer; (k) sodium or a salicylic acid derivative; (l) a glycerol ester of acetoacetic acid; (m) a cyclodextrin or beta-cyclodextrin derivative; (n) a medium-chain fatty acid; (o) a chelating agent; (p) an amino acid or salt thereof; (q) an N-acetylamino acid or salt thereof; and (r) an enzyme degradative to a selected membrane component. In some embodiments, the compositions described herein further comprise a delivery enhancing agent.

### Sublingual Formulations

Also provided herein are sublingual compositions comprising phenothiazines or derivatives thereof. The sublingual compositions described herein comprise (a) a phenothiazine or derivative thereof, (b) one or more binders, (c) one or more soluble agents, (d) one or more disintegrants, (e) a pH-control agent, (f) one or more lubricants and (g) one or more sweeteners and/or flavorings. In some embodiments, the sublingual compositions are solid compositions. In some other embodiments, the sublingual compositions are liquid compositions. In some embodiments, the sublingual compositions described herein are tablets, capsules, lozenges, gums, or films.

### Phenothiazines

Phenothiazines suitable for the compositions described herein include, piperadine and piperazine phenothiazines, e.g., Chlorpromazine, Promazine, Triflupromazine, Methotrimeprazine, Mesoridazine, Thioridazine, Fluphenazine, Perphenazine, Flupentixol, Prochlorperazine, Trifluoperazine. Promethazine, thioridazine, and Acetophenazine. In some embodiments, the phenothiazine is prochlorperazine. In some instances, the compositions described herein comprise suitable derivatives of phenothiazines. In some embodiments, derivatives of phenothiazines allow for enhanced absorption across a nasal mucosa. Suitable derivatives of phenothiazines include, e.g., lipid conjugates and/or prodrugs e.g., ester derivatives of phenothiazines. Additional derivatives include pharmaceutically acceptable salts, solvates, isomers, tautomers, metabolites, analogs, or prodrugs thereof.

Phenothiazines such as, for example, promethazine, prochlorperazine, thioproperazine, fluopromazine, perphenazine, are used in the treatment of nausea and vomiting. In some instances, the treatment of nausea or emesis requires administration of multiple doses within a 24 hour period. For example, the current recommended dosage of prochlorperazine in the treatment of nausea and/or emesis is: Oral Dosage Tablets 5 mg or 10 mg tablet 3 or 4 times daily; Rectal Dose 25 mg twice daily; Intramuscular dose initial dose of 5 to 10 mg (1 to 2 mL) injected deeply into the upper outer quadrant of the buttock, with a repeat dose every 3 or 4 hours; Intravenous dose 2½ to 10 mg (½ to 2 mL) by slow I.V. injection.

The administration of conventional doses of phenothiazines is associated with side effects including, drowsiness, dizziness, blurred vision, abnormal menstrual flow, rash, low blood pressure, and/or distorted voluntary movements of the body (dystonic reaction). In some instances, cholestatic jaundice or fever with grippe-like symptoms may occur. Depending on the severity of side effects and symptoms, dosage is reduced or discontinued. If therapy is reinstituted, it is at a lower dosage. If side effects and symptoms persist, the drug is stopped and not reinstituted.

The administration of phenothiazines such as prochlorperazine via an intranasal or sublingual route allows for delivery of the phenothiazine directly into the circulatory system, avoiding drug degradation and concomitant side effects in the gastrointestinal (GI) tract. The intranasal or sublingual route of administration bypasses first-pass metabolism and provides an alternate route of administration for phenothiazines that have erratic oral or parenteral administration pharmacokinetics. Intranasal or sublingual administration allows for low dosage frequency and lower doses, which minimizes adverse reactions associated with phenothiazine treatment.

### Alkyl Glycoside

The term “alkyl glycoside” refers to a sugar joined by a linkage to a hydrophobic alkyl group. The hydrophobic alkyl group in an alkyl glycoside is an alkyl group containing from 9 to 24 carbon atoms and is of any desired size, depending on the hydrophobicity desired and depending on the hydrophilicity of the saccharide moiety that it is attached to. In some embodiments, the alkyl chain is from 9 to 14 carbon atoms.

Alkyl glycosides that are compatible with the compositions disclosed herein include: alkyl glycosides, such as octyl-, nonyl-, decyl-, undecyl-, dodecyl-, tridecyl-, tetradecyl, pentadecyl-, and octadecyl α- or β-D-maltoside, -glucoside or -sucroside, alkyl thiomaltosides, such as heptyl, octyl, dodecyl-, tridecyl-, and tetradecyl-.β-D-thiomaltoside; alkyl thioglucosides, such as heptyl- or octyl 1-thio α- or β-D-glucopyranoside; alkyl thiosucroses; alkyl maltotriosides; long chain aliphatic carbonic acid amides of sucrose β-amino-alkyl ethers; derivatives of palatinose and isomaltamine linked by amide linkage to an alkyl chain; derivatives of isomaltamine linked by urea to an alkyl chain; long chain aliphatic carbonic acid ureides of sucrose, β-amino-alkyl ethers; and long chain aliphatic carbonic acid amides of sucrose β-amino-alkyl ethers.

In some embodiments, alkyl glycosides include maltose, sucrose, and glucose linked by glycosidic linkage to an alkyl chain of 9, 10, 12 or 14 carbon atoms, i.e., nonyl-, decyl-, dodecyl- and tetradecyl sucroside, glucoside, and maltoside. In some embodiments, the alkyl glycoside is dodecyl maltoside, tridecyl maltoside, tetradecyl malto side, sucrose mon-dodecanoate, sucrose mono-tridecanoate and sucrose mono-tetradecanoate.

In some instances, the formulations described herein comprise alkyl glycosides in the range of about 0.01% to about 10%. In some instances, the formulations described herein comprise alkyl glycosides in the range of about 0.01 to about 2%, 4% or 5%. In some instances, the formulations described herein comprise alkyl glycosides in the range of about 0.1 to about 2%, 3%, 4% or 5%; from about 0.5% to 1%, 2%, 3%, 4%, 5%, 6%, 7%, 8% or 9%; from about 1% to about 2%, 3%, 4%, 5%, 6%, 7%, 8% or 9%; from about 2.5%, to about 4%, 5%, 6%, 7%, 8% or 9%; or from about 5% to about 9%. In some instances, the formulations described herein comprise alkyl glycosides in the range of about 0.01% to about 6%.

The term “saccharide” is inclusive of monosaccharides, oligosaccharides or polysaccharides in straight chain or ring forms. Oligosaccharides are saccharides having two or more monosaccharide residues. Non-limiting examples of saccharides include glucose, maltose, maltotriose, maltotetraose, sucrose and trehalose.

### Permeation Enhancers

“Surfactants” are wetting agents that lower the surface tension of a liquid. Surfactants that are suitable for the compositions described herein include but are not limited to: a) natural and synthetic lipophilic agents, e.g., phospholipids, cholesterol, and cholesterol fatty acid esters and derivatives thereof; b) nonionic surfactants, which include for example, polyoxyethylene fatty alcohol esters, sorbitan fatty acid esters (Spans), polyoxyethylene sorbitan fatty acid esters (e.g., polyoxyethylene (20) sorbitan monooleate (Tween® 80), polyoxyethylene (20) sorbitan monostearate (Tween® 60), polyoxyethylene (20) sorbitan monolaurate (Tween® 20) and other Tweens, sorbitan esters, glycerol esters, e.g., Myrj® and glycerol triacetate (triacetin), polyethylene glycols, cetyl alcohol, cetostearyl alcohol, stearyl alcohol, polysorbate 80, poloxamers, Carbopols®, poloxamines, polyoxyethylene castor oil derivatives (e.g., CREMOPHOR® RH40, CREMOPHOR® A25, CREMOPHOR® A20, CREMOPHOR® EL) and other Cremophors, sulfosuccinates, alkyl sulphates (SLS); PEG glyceryl fatty acid esters such as PEG-8 glyceryl caprylate/caprate (Labrasol®), PEG-4 glyceryl caprylate/caprate (Labrafac Hydro WL 1219), PEG-32 glyceryl laurate (Gelucire® 444/14), PEG-6 glyceryl mono oleate (Labrafil® M 1944 CS), PEG-6 glyceryl linoleate (Labrafil® M 2125 CS); propylene glycol mono- and di-fatty acid esters, such as propylene glycol laurate, propylene glycol caprylate/caprate; BRIJ® 700, ascorbyl-6-palmitate, stearylamine, sodium lauryl sulfate, polyoxethyleneglycerol triricinoleate, and any combinations or mixtures thereof; c) anionic surfactants include, but are not limited to, calcium carboxymethylcellulose, sodium carboxymethylcellulose, sodium sulfosuccinate, dioctyl, sodium alginate, alkyl polyoxyethylene sulfates, sodium lauryl sulfate, triethanolamine stearate, potassium laurate, bile salts, and any combinations or mixtures thereof; and d) cationic surfactants such as cetyltrimethylammonium bromide, and lauryldimethylbenzyl-ammonium chloride.

The nasal formulations described herein comprise surfactants at a level of from about 0.01% to 20% by weight. In some instances, the nasal formulation described herein comprise surfactant from about 0.01% to 5% by weight, from about 0.01% to 2% by weight, from about 0.01% to 1%, or from about 0.01% to 0.125% by weight of the formulation. The concentration of the surfactant in the formulation is optimized such that the concentration of the penetration enhancer is as low as possible, while still maintaining the desired effect.

Bile acid salts that are suitable in the compositions described herein include for example, salts of taurocholic acid, glycocholic acid, cholic acid, chenodeoxycholic acid, deoxycholic acid, and lithocholic acid. Bile acid conjugates, e.g. glycine and taurine conjugates, sodium tauro-24,25-dihydrofusidate (STDHF), are also suitable for the compositions described herein.

In some embodiments, the compositions described herein comprise a cyclodextrin. Cyclodextrins are cyclic oligosaccharides containing 6, 7, or 8 glucopyranose units, referred to as α-cyclodextrin, β-cyclodextrin, or γ-cyclodextrin respectively. Cyclodextrins have a hydrophilic exterior, which enhances water-solubility, and a hydrophobic interior, which forms a cavity. In an aqueous environment, hydrophobic portions of other molecules often enter the hydrophobic cavity of cyclodextrin to form inclusion compounds. Additionally, cyclodextrins are capable of other types of nonbonding interactions with molecules that are not inside the hydrophobic cavity. Cyclodextrins have three free hydroxyl groups for each glucopyranose unit, or 18 hydroxyl groups on α-cyclodextrin, 21 hydroxyl groups on β-cyclodextrin, and 24 hydroxyl groups on γ-cyclodextrin. One or more of these hydroxyl groups can be reacted with any of a number of reagents to form a large variety of cyclodextrin derivatives, including hydroxypropyl ethers, sulfonates, and sulfoalkylethers. Shown below is the structure of β-cyclodextrin and the hydroxypropyl-β-cyclodextrin (HPβCD).

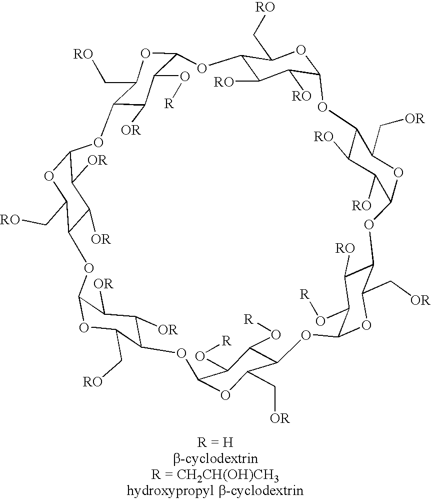

In some embodiments, the use of cyclodextrins in the pharmaceutical compositions described herein improves the solubility of the drug. Inclusion compounds are involved in many cases of enhanced solubility; however other interactions between cyclodextrins and insoluble compounds can also improve solubility. Hydroxypropyl-β-cyclodextrin (HPβCD) is commercially available as a pyrogen free product. It is a nonhygroscopic white powder that readily dissolves in water. HPβCD is thermally stable and does not degrade at neutral pH. Thus, cyclodextrins improve the solubility of a therapeutic agent in a composition or formulation. Accordingly, in some embodiments, cyclodextrins are included to increase the solubility of the otic agent within the formulations described herein. In other embodiments, cyclodextrins in addition serve as controlled release excipients within the formulations described herein.

By way of example only, cyclodextrin derivatives suitable for use in formulations described herein include α-cyclodextrin, β-cyclodextrin, γ-cyclodextrin, hydroxyethyl β-cyclodextrin, hydroxypropyl γ-cyclodextrin, sulfated β-cyclodextrin, sulfated α-cyclodextrin, sulfobutyl ether β-cyclodextrin.

In some embodiments, the compositions described herein comprise liposomes. Liposomes are spheres of phospholipid which encapsulate the deliverable contents (e.g., a phenothiazine). Liposomes protect the encapsulated content and due to their phospholipid composition, are able to pass across epithelial barriers (e.g., mucosal membranes) into the blood stream. In some embodiments, a permeation enhancer is a mixed micelle.

In some embodiments, a permeation enhancer loosens junctions in a barrier such as a mucosal membrane. By way of non-limiting example, chelating agents (e.g., EDTA) remove metal ions (e.g., calcium) from cell junctions and facilitate passage of a phenothiazine or a derivative thereof, and/or a liposome encapsulating a phenothiazine or derivative thereof through a nasal membrane.

In some embodiments, a permeation enhancer is an enzyme degradative to a selected membrane component, e.g., glycans in/on cell membranes. An enzyme degradative to a membrane component includes, by way of example, a glycosidase, a heparanase, a hyaluronidase, a keratinase, or combinations thereof.

In some embodiments, a permeation enhancer is a NO donor compound that provides for the release of nitric oxide for passage across the epithelial monolayer of a membrane (e.g., a nasal membrane) thereby causing local vasodilation without causing a similar vasodilation of the systemic vasculature. Exemplary NO donors include, e.g., S-nitroso-N-acetyl-DL-penicillamine, NOR1, NOR4-which are preferably co-administered with an NO scavenger such as carboxy-PITO or doclofenac sodium. Further NO donor compounds include, by way of non-limiting example, compounds disclosed in WO/2002/041924, U.S. Pat. No. 5,958,427, US Patent Appl. Pub. Nos. 2008/0233163, 2006/0183913, NO donor compounds that are disclosed therein are hereby incorporated by reference herein.

In some embodiments, a permeation enhancer is an alkyl glycoside as described herein.

### Delivery Enhancing Agents

In some embodiments, the compositions described herein comprise a delivery enhancing agent. In some instances, the delivery enhancing agent is chitosan, a carboxymethylated chitosan, or chitin, and alginate. In some embodiments, a combination of carboxymethylated chitosan and alginate is optionally used to assist in increasing the permeability of the phenothiazine through the nasal mucosa. Additional examples include, but are not limited to, Poly(acrylic acid)-elastatinal (anti-elastase), Carboxymethylcellulose-elastatinal (anti-elastase), Polycarbophil—elastatinal (anti-elastase), Chitosan—antipain (anti-trypsin), Poly(acrylic acid)—bacitracin (anti-aminopeptidase N), Chitosan—EDTA (anti-aminopeptidase N, anti-carboxypeptidase A), Chitosan—EDTA—antipain (anti-trypsin, anti-chymotrypsin, anti-elastase). Further contemplated herein are novel chitosan derivative or chemically modified form of chitosan. By way of example, a chitosan derivative is β-[1→4]-2-guanidino-2-deoxy-D-glucose polymer (poly-GuD).

In some embodiments, the delivery enhancing agent is a degradative enzyme inhibitory agent. In some embodiments, the degradative enzyme is a protease, (e.g., a metalloproteinase (e.g. MMP1), a serine protease, a cysteine protease), an esterase (e.g., a lipase, a phosphatase), a hyaluronidase or the like. In some embodiments, a degradative enzyme inhibitory agent is a protease inhibitor. In some embodiments, the delivery enhancing agent is a mucolytic or mucus clearing agent, including, by way of non-limiting example, Acetylcysteine, Ambroxol, Bromhexine, Carbocisteine, Domiodol, Dornase alfa, Eprazinone, Erdosteine, Letosteine, Mesna, Neltenexine, Sobrerol, Stepronin, and Tiopronin.

In some embodiments, a delivery enhancing agent is a charge-modifying agent. A charge modifying agent modifies the charge on a membrane (e.g., nasal membrane) via protonation, deprotonation and/or cross linking of charged moieties to a membrane. In some instances, a charge-modifying agent is a cationic charge-modifying agent. In some instances a cationic charge-modifying agent is a polyamine or an aziridine-ethylene oxide copolymer. In some instances, the polyamine is a polyethyleneimine and like polyamines. In some instances, a cationic charge-modifying agent is a water soluble organic polymer having a molecular weight greater than about 1000 daltons, wherein the polymer has at least one epoxide or epichlorohydrin substituent capable of bonding to a membrane and wherein the polymer also has at least one tertiary amine or quaternary ammonium group capable of providing a cationic charge site.

In some instances, a delivery enhancing agent is a ciliostatic agent. Ciliostatic agents include, by way of example, benzalkonium chloride, EDTA, and/or bile salts. Various bacterial ciliostatic factors isolated and characterized in the literature may be employed within these embodiments of the invention. Non-limiting examples of ciliostatic factors include those from the bacterium Pseudomonas aeruginosa such as a phenazine derivative, a pyo compound (2-alkyl-4-hydroxyquinolines), and a rhamnolipid (also known as a hemolysin). Additional ciliostatic agents are known in the art and contemplated herein.

The mucosal therapeutic and prophylactic compositions of the present invention may be supplemented with any suitable penetration-promoting agent that facilitates absorption, diffusion, or penetration across mucosal barriers. The penetration promoter may be any promoter that is pharmaceutically acceptable. Additional delivery enhancing agents and/or permeation enhancers can include, but are not limited to, sodium salicylate and salicylic acid derivatives (acetyl salicylate, choline salicylate, salicylamide, etc.); amino acids and salts thereof (e.g., monoaminocarboxylic acids such as glycine, alanine, phenylalanine, proline, hydroxyproline, etc.; hydroxyamino acids such as serine; acidic amino acids such as aspartic acid, glutamic acid, etc.; and basic amino acids such as lysine etc—inclusive of their alkali metal or alkaline earth metal salts); and N-acetylamino acids (N-acetylalanine, N-acetylphenylalanine, N-acetylserine, N-acetylglycine, N-acetylglycine, N-acetylglutamic acid, N-acetylproline, N-acetylhydroxyproline, etc.) and their salts (alkali metal salts and alkaline earth metal salts). Also provided as penetration-promoting agents within the methods and compositions of the invention are substances which are generally used as emulsifiers (e.g., sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and the like.

### Soluble Agents

Soluble agents accelerate the solubilization of a sublingual composition described herein. A soluble agent is selected from sugars such as sucrose, lactose or dextrose, of polyols such as mannitol, sorbitol or lactitol, or the like, or else inorganic salts such as sodium chloride or the like.

### Binder

A binder “bonds” an active substance and other excipients. Suitable binder materials include, but are not limited to, starch (e.g., corn starch), pregelatinized starch, gelatin, sugars (including sucrose, glucose, dextrose, lactose and sorbitol), dextrin, maltodextrin, zein, polyethylene glycol, waxes, natural and synthetic gums such as acacia, guar gum, tragacanth, alginate, sodium alginate, celluloses, including hydroxypropylmethylcellulose, carboxymethylcellulose sodium, hydroxypropylcellulose, hydroxylethylcellulose, ethylcellulose, methyl cellulose, and veegum, hydrogenated vegetable oil, Type I, magnesium aluminum silicate, and synthetic polymers such as acrylic acid and methacrylic acid copolymers, carbomer, methacrylic acid copolymers, methyl methacrylate copolymers, aminoalkyl methacrylate copolymers, polyacrylic acid/polymethacrylic acid, and polyvinylpyrrolidone.

### Lubricants

Lubricants are used to facilitate tablet manufacture. Examples of suitable lubricants include, but are not limited to, magnesium stearate, calcium stearate, stearic acid, glyceryl behenate, glyceryl monostearate, glyceryl palmitostearate, hydrogenated castor oil, hydrogenated vegetable oil, type I, sodium benzoate, sodium lauryl sulfate, sodium stearyl fumarate, polyethylene glycol, talc, zinc stearate, and mineral oil and light mineral oil.

### Disintegrants

Disintegrants are used to facilitate dosage form disintegration or “breakup” after administration, and generally include, but are not limited to, starch, sodium starch glycolate, sodium carboxymethyl starch, methylcellulose, calcium carboxymethylcellulose, sodium carboxymethylcellulose, hydroxypropyl cellulose, microcrystalline cellulose, colloidal silicon dioxide, croscarmellose sodium (e.g., Ac-Di-Sol croscarmellose sodium), pregelatinized starch, clays, cellulose, powdered cellulose, pregelatinized starch, sodium starch glycolate, sodium aginate, alginic acid, guar gum, magnesium aluminum silicate, polacrilin potassium, and cross linked polymers, such as cross-linked PVP, crospovidone (POLYPLASDONE® from GAF Chemical Corp). Disintegrants allow for rapid release of a phenothiazine from a sublingual formulation.

As generally used herein, an immediate release sublingual formulation is considered “rapidly dissolving” when no less than 85% of the amount of the drug substance dissolves within 30 minutes, using U.S. Pharmacopeia (USP) Apparatus I at 100 rpm (or Apparatus II at 50 rpm) in a volume of 900 ml or less in each of the following media: (1) 0.1 N HCl or Simulated Gastric Fluid USP without enzymes; (2) a pH 4.5 buffer; and (3) a pH 6.8 buffer or Simulated Intestinal Fluid USP without enzymes.

### pH Control Agents

In some embodiments, the nasal and/or sublingual compositions described herein also include one or more pH control agents or buffering agents. Suitable pH control agents or buffers include, but are not limited to acetate, bicarbonate, ammonium chloride, citrate, phosphate, pharmaceutically acceptable salts thereof and combinations or mixtures thereof. Such pH adjusting agents and buffers are included in an amount required to maintain pH of the composition between a pH of about 4.5 and about 9, between a pH of about 5 and about 8, in one embodiment a pH between about 4.5 to about 6.0; in one embodiment a pH between about 6.5 to about 7.5, and in yet another embodiment at a pH of about 6.5, 6.6, 6.7, 6.8, 6.9, 7.0, 7.1, 7.2, 7.3, 7.4, 7.5, 7.6, or 7.8. In one embodiment, when one or more buffers are utilized in the formulations of the present disclosure, they are combined, e.g., with a pharmaceutically acceptable vehicle and are present in the final formulation, e.g., in an amount ranging from about 0.1% to about 20%, from about 0.5% to about 10%. In certain embodiments of the present disclosure, the amount of buffer included in the gel formulations is an amount such that the pH of the gel formulation does not interfere with the nasal membrane's natural buffering system. In some embodiments, from about 5 mM to about 200 mM concentration of a buffer is present in the gel formulation. In certain embodiments, from about a 20 mM to about a 100 mM concentration of a buffer is present. In one embodiment is a buffer such as acetate or citrate at slightly acidic pH. In one embodiment the buffer is a sodium acetate buffer having a pH of about 4.5 to about 6.5. In one embodiment the buffer is a sodium citrate buffer having a pH of about 5.0 to about 8.0, or about 5.5 to about 7.0.

In an alternative embodiment, the buffer used is tris(hydroxymethyl)aminomethane, bicarbonate, carbonate or phosphate at slightly basic pH. In one embodiment, the buffer is a sodium bicarbonate buffer having a pH of about 6.5 to about 8.5, or about 7.0 to about 8.0. In another embodiment the buffer is a sodium phosphate dibasic buffer having a pH of about 6.0 to about 9.0.

In some embodiments, the pharmaceutical formulations described herein are stable with respect to pH over a period of any of at least about 1 day, at least about 2 days, at least about 3 days, at least about 4 days, at least about 5 days, at least about 6 days, at least about 1 week, at least about 2 weeks, at least about 4 weeks, at least about 6 weeks, at least about 8 weeks, at least about 4 months, at least about 5 months, or at least about 6 months. In other embodiments, the formulations described herein are stable with respect to pH over a period of at least about 1 week to about 1 month. Also described herein are formulations that are stable with respect to pH over a period of at least about 1 month.

In some instances, sublingual compositions described herein comprise a pH-modifier that is chosen from an acid base pair. The acid base pair is selected from citric acid and sodium citrate or potassium citrate, sodium hydroxide, monoethanolamine, diethanolamine, sodium bicarbonate or potassium bicarbonate, sodium phosphate, tartaric acid, propionic acid, lactic acid, malic acid and monosodium glutamate. The acid/base pair that produces an effervescence in the presence of water and/or saliva, and allows release of a gas. The acidic agent can consist of any inorganic or organic acid, in the form of a free acid, an acid anhydride or an acid salt. The acid is chosen from the group comprising tartaric acid, citric acid, maleic acid, fumaric acid, malic acid, adipic acid, succinic acid, lactic acid, glycolic acid, alpha-hydroxy acids, ascorbic acid and amino acids, and also the salts and derivatives of these acids. The alkaline agent consists of a compound capable of generating a gas by reaction with a proton-donating compound. By way of non-limiting example, an alkaline agent is chosen from the group comprising potassium carbonate, lithium carbonate, sodium carbonate, calcium carbonate, ammonium carbonate, L-lysine carbonate, arginine carbonate, sodium glycine carbonate, sodium carbonates of amino acids, anhydrous sodium perborate, effervescent perborate, sodium perborate monohydrate, sodium percarbonate, sodium dichloroisocyanurate, sodium hypochlorite, calcium hypochlorite, and mixtures thereof. The gas formed is carbon dioxide, oxygen or any other type of biocompatible gas.

The effervescent mixture facilitates the rapid dissolution of sublingual composition upon contact with saliva, and thus obtaining, through the release of a pharmaceutically acceptable gas and induction of a buccal micro pH, rapid solubilization of the active substance at the sublingual mucous membranes and an improved systemic passage while at the same time improving the organoleptic properties so as to decrease the feeling of the active substance in the buccal cavity, or inducing a pleasant slightly acid taste. Suitable sweeteners are aspartame, acesulfam potassium, sodium saccharinate, neohesperidine dihydrochalcone, sucralose, monoammonium glycyrrhizinate, and mixtures thereof.

### Additional Excipients

In some embodiments, the nasal and/or sublingual compositions described herein comprise additional excipients, including gels that modulate the viscosity of the compositions. Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums, (e.g., tragacanth). In some embodiments, single-phase gels are generally aqueous, but will also be made using alcohols and oils. Two-phase gels consist of a network of small discrete particles.

Gels can also be classified as being hydrophobic or hydrophilic. In certain embodiments, the base of a hydrophobic gel consists of a liquid paraffin with polyethylene or fatty oils gelled with colloidal silica, or aluminum or zinc soaps. In contrast, the base of hydrophobic gels usually consists of water, glycerol, or propylene glycol gelled with a suitable gelling agent (e.g., tragacanth, starch, cellulose derivatives, carboxyvinyl polymers, and magnesium-aluminum silicates).

In some instances, the rate of diffusion of a therapeutic agent (e.g., a phenothiazine) decreases with increasing polarity, increasing molecular weight or increasing viscosity of the nasal formulation. The application of a thinner composition to the nasal mucosal membranes minimizes the effects diffusion gradients. A higher concentration of the therapeutic agent in increases the rate of diffusion across the nasal mucosal membranes. The choice of an appropriate gel excipient allows for control of the absorption of a therapeutic agent across a nasal membrane. The viscosity of the compositions described herein range from about. 00001 centipoise to about 10,000 centipoise and contain from about. 000001% to about 25% by weight of one or more phenothiazines. The compositions described herein are topically applied to the nasal mucosal membranes as aerosols, liquid drops or gels.

Polymers and/or gels that are suitable for use in compositions described herein include and are not limited to polymers composed of polyoxypropylene and polyoxyethylene and polyoxyethylene-polyoxypropylene copolymers. celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum, glycerin-based gels (e.g., Elta® Hydrogel (Swiss-American Products, Inc., Dallas, Tex.) and K-Y® Sterile (Johnson & Johnson), glycerin-derived compounds, conjugated, or crosslinked gels, matrices, hydrogels, as well as gelatins and their derivatives, alginates, and alginate-based gels (e.g., alginate hydrogels SAF-Gel® (ConvaTec, Princeton, N.J.), and even various native and synthetic hydrogel and hydrogel-derived compounds and any combinations or mixtures thereof. In some embodiments, Chitosan glycerophosphate (CGP) is used as an excipient for the formation of hydrogels

Other excipients that are useful in the nasal and/or sublingual compositions disclosed herein include stabilizers including, for example, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, and combinations thereof. In some embodiments, amide analogues of stabilizers are also used. In further embodiments, the chosen stabilizer changes the hydrophobicity of the formulation (e.g., oleic acid, waxes), or improves the mixing of various components in the formulation (e.g., an alcohol such as ethanol, isopropanol or the like, or a polyol such as propylene glycol, polyethylen glycol or the like), and/or controls the moisture level in the formula (e.g., PVP or polyvinyl pyrrolidone).

The compositions described herein optionally include one or more antioxidant stabilizers to enhance chemical stability where required. Suitable antioxidants include, by way of example only, ascorbic acid, methionine, sodium thiosulfate and sodium metabisulfite. In one embodiment, antioxidants are selected from metal chelating agents, thiol containing compounds and other general stabilizing agents. In some embodiments, the compositions described herein comprise preservatives, including, but are not limited to, benzoic acid, boric acid, p-hydroxybenzoates, alcohols, quarternary compounds, stabilized chlorine dioxide, mercurials, such as merfen and thiomersal, mixtures of the foregoing and the like. Other suitable stabilizers include polysaccharides, such as cellulose and cellulose derivatives, and simple alcohols, such as glycerol, lecithins, such as natural lecithins (e.g. egg yolk lecithin or soya bean lecithin) and synthetic or semisynthetic lecithins (e.g. dimyristoylphosphatidylcholine, dipalmitoylphosphatidylcholine or distearoyl-phosphatidylcholine; phosphatidic acids; phosphatidylethanolamines; phosphatidylserines such as distearoyl-phosphatidylserine, dipalmitoylphosphatidylserine and diarachidoylphospahtidylserine; phosphatidylglycerols; phosphatidylinositols; cardiolipins; sphingomyelins; and synthetic detergents, such as diosctanoylphosphatidyl choline and polyethylene-polypropylene glycol), acacia, albumin, alginic acid, bentonite, carboxymethylcellulose calcium, carboxymethylcellulose sodium, cyclodextrins, glyceryl monostearate, hydroxypropyl cellulose, hydroxypropyl methylcellulose, magnesium aluminum silicate, propylene glycol, propylene glycol alginate, sodium alginate, white wax, xanthan gum, and yellow wax.

Other excipients that are optionally included in the nasal and/or sublingual compositions described herein include one or more diluents. Salts dissolved in buffered solutions (which also can provide pH control or maintenance) are utilized as diluents, including, but not limited to a phosphate buffered saline solution. In other embodiments, isotonic formulations are provided by the addition of a tonicity agent. Suitable tonicity agents include, but are not limited to any pharmaceutically acceptable sugar, salt or any combinations or mixtures thereof, such as, but not limited to dextrose and sodium chloride.

In some embodiments, any pharmaceutical composition described herein is stable with respect to phenothiazine degradation over a period of any of at least about 1 week, at least about 2 weeks, at least about 3 weeks, at least about 4 weeks, at least about 5 weeks, at least about 6 weeks, at least about 7 weeks, at least about 8 weeks, at least about 3 months, at least about 4 months, at least about 5 months, or at least about 6 months. In other embodiments, the formulations described herein are stable with respect to compound degradation over a period of at least about 1 week. Also described herein are formulations that are stable with respect to compound degradation over a period of at least about 1 month. Also described herein are formulations that are stable with respect to compound degradation over a period of at least about 2 months. Also described herein are formulations that are stable with respect to compound degradation over a period of at least about 6 months.

The concentration of a permeation enhancer, alkyl glycoside, delivery enhancer and/or other excipients used in any composition and method disclosed herein can vary according to the physiochemical properties, pharmacokinetic properties, side effect or adverse events, formulation considerations, or other factors associated with the therapeutically phenothiazine or with the properties of other excipients in the composition. Thus, in certain circumstances, the concentration or amount of the permeation enhancer, alkyl glycoside, delivery enhancer and/or other excipients used in accordance with the compositions and methods disclosed herein will vary, depending on the need.

### Nasal Administration

In some embodiments, provided herein are nasal phenothiazine dosages. Nasal dosages are administered as a nasal spray or nasal drop. By way of example, nasal sprays or drops comprise a pharmaceutically acceptable carrier or excipient. Nasal sprays may be liquid or solid nasal sprays. The nasal sprays may be aerosol or non-aerosol nasal sprays. The nasal delivery system is for example an aerosolized metered dose pump, a manual metered dose pump, or a metered dose spray-producing squeeze bottle. In some embodiments, the nasal delivery system is an aerosolized metered dose pump.

Spray formulations are prepared by dissolving or suspending the phenothiazine in a pharmaceutically acceptable carrier or excipient. By way of example, pharmaceutically acceptable carriers or excipients include liquid medium that may also contain other ingredients such as surfactants, stabilising agents, buffering agents, flavourings, sweeteners, colouring agents and preservatives. For example, a spray may be prepared by dissolving water soluble components in water and non-water soluble ingredients in a co-solvent (e.g. an alcohol). The two phases are then mixed and the resulting mixture filtered and placed into dispensing containers. Taste enhancers and/or flavoring agents, stabilising agents, buffering agents, and preservatives are optionally added to the compositions. Suitable solubilizers and auxiliary solubilizers capable of forming aqueous solutions include by way of example, distilled water for solution, physiological saline and propylene glycol. Suspending agents (e.g., surfactant such as polysorbate 80), pH regulators (e.g., organic acid and metal salt thereof) and stabilizers are also used for nasal sprays. Compositions suitable for nasal administration described herein include sterile suspensions, emulsions or sterile solutions of the phenothiazine in a suitable solvent.

The term aerosol refers to a suspension or dispersion of either liquid droplets or solid powder in air. In this context, liquid droplets may be formed from solutions, suspensions and dispersions of drug in a liquid medium, such as water or a non-aqueous medium. The liquid medium may also contain one or more diluents, excipients, enhancers or additional active pharmaceutical ingredients. Where the aerosol is a suspension of liquid in air, it is possible, and in some embodiments of the invention preferred, that the liquid contain particles of a drug compound that are insoluble or slightly soluble in the liquid. It is also possible for the drug to be fully soluble in the liquid.

Solid powder includes solid particulates comprising solid drug and optionally one or more non-liquid diluents, excipients, and/or diluents.

An aerosol is insufflated using a suitable mechanical apparatus. The dispensing containers may be fitted with a metered, manually-operated spray mechanism or the dispenser may contain a pressurized propellant and be fitted with a suitable dispensing valve. The spray and dispensing container may be adapted for nasal administration of the spray, for example a sterile suspension or sterile solution of the phenothiazine.

In some embodiments, the apparatus may include a reservoir and sprayer, which is a device adapted to expel the pharmaceutical dose in the form of a spray. A number of doses of the drug to be administered may be contained within the reservoir, optionally in a liquid solution or suspension or in a solid particulate formulation, such as a solid particulate mixture.

In some embodiments, the apparatus is a pump sprayer that includes a metering pump. In some embodiments, the apparatus includes a pressurized spray device, in which the sprayer includes a metering valve and the pharmaceutical composition further comprises a pharmaceutically acceptable propellant. Exemplary propellants include one or mixture of chlorofluorocarbons, such as dichlorodifluoromethane, as well as the currently preferred hydrofluorocarbons, such as 1,1,1,2-tetrafluoroethane (HFC-134a) and 1,1,1,2,3,3,3-heptafluoropropane (HFC-227).

In some embodiments, the apparatus is a nebulizer that comprises tubing connected to a compressed air source which causes air or oxygen to blast at high velocity through a liquid solution to turn it into an aerosol, which is then inhaled by the patient

In some embodiments, powders can be administered using a nasal insufflator. In some embodiments, powders may be contained within a capsule, which is inserted into an insufflation device. The capsule is punctured by a needle, which makes apertures at the top and bottom of the capsule. Air or other pharmaceutically acceptable propellant is then sent through the needle to blow out powder particles. In some embodiments, pharmaceutically acceptable propellants include ethyl chloride, butane, propane, dichlorodifluoromethane, dichlorotetrafluoroethane, and trichloromonofluoromethane.

A “intranasal metered dose sprayer” is a device for repeated delivery of defined quantities of a drug product through the intranasal route. Such devices are broadly available and modes of their use are well-known in the pharmaceutical field. One example of an intranasal metered dose sprayer is described in International Patent Publication No. WO 02/13886, designating the United States, which is incorporated herein by reference in its entirety. The device of WO 02/13886 is programmable and provides individual vials for each dose. Another metered dose sprayer for intranasal use for delivery of multiple single doses of a powder from a single vial is described in U.S. Pat. No. 6,055,979. Lastly, metered dose sprayers for intranasal use are broadly available commercially, for example, from Pfeiffer GmbH of Radolfzell, Germany.

In some embodiments, the intranasal compositions described herein comprise particles with an average diameter ranging from 1 μm to 10 μm. In some instances, the intranasal compositions described herein comprise particles with an average diameter ranging from 2 μm to 5 μm. In some instances, the intranasal compositions described herein comprise particles with an average diameter ranging from 10 nm to 1 μm, from 10 nm to 500 nm, or from 10 nm to 100 nm. In some embodiments, the intranasal composition described herein comprise micronized particles.

Also provided herein is a method of making a pharmaceutical composition for nasal delivery comprising formulating a pharmaceutical composition comprising at least one phenothiazine or phenothiazine derivative and at least one permeation enhancer, wherein said pharmaceutical composition is suitable for nasal administration.

### Sublingual Administration

The sublingual compositions described herein may be in the form of a dry powder containing the pharmaceutically phenothiazine and one or more excipient(s). The phenothiazine and excipient may be stored together or separately. The phenothiazine and excipients may be stored together if the phenothiazine is stable in the presence of the excipients. Alternatively, they may be stored separately, and then mixed before, during or after they are dispensed to the oral cavity. The dry powder rapidly dissolves upon mixing with saliva and effectively delivers the phenothiazine to the systemic circulation via absorption through the sublingual epithelium. In some embodiments, the dry powder sublingual compositions described herein are formulated for rapid release (e.g., by reducing particle size).

The phenothiazines and excipients may be in the form of particles having the same or different sizes. In one embodiment, the excipient particles are larger than the particles of the phenothiazine. The small particles of a phenothiazine coat the larger particle so that both particles are administered simultaneously. For sublingual delivery, the large particles have diameters greater than 8 μm, and range from 8 μm to 500 μm, and small particles have a diameter ranging from about 1 nm to 9 μm. In some instances, small particles have a diameter ranging from about 100 nm to 400 nm.

Optionally, the particles are oppositely charged, so that the excipient particles contain one charge and the phenothiazine particles contain the opposite charge so that the particles are administered simultaneously. The charged particles may be formed for example, by using an acidic solution to make one of the particles, and a basic solution to form the other particles. If the particles of agent and excipient are oppositely charged, they may have the same average diameter or different average diameters.

In some embodiments, the sublingual compositions described herein are the form of a film with a thickness range from 0.01 to 2 mm. The film may have any suitable shape, including round, oval, rectangle, or square. The film may be a monolayer, bilayer or trilayer film. Each of the layers may be different, or two of the layers, such as the bottom and top layers, may have substantially the same composition. In one embodiment, the bottom and top layers surround a core layer containing the phenothiazine. The bottom and top layers may contain one or more excipients, such as a solubilizing agent and a metal chelator.

In some instances, the sublingual compositions described herein are formulated for rapid release. By altering the composition of the excipients, a film is designed to dissolve rapidly (less than 30 seconds) or slowly (up to 15 minutes) in order to achieve the desired absorption profile and subsequent effect. The film may dissolve in a time period ranging from 3 to 5 minutes, 5 to 8 minutes, or 8 to 12 minutes or 15 seconds to 2 minutes.

In some embodiments, the sublingual compositions described herein are in the form of a lozenge, tablet, capsule, or wafer containing the phenothiazine and one or more excipients, such as chelators, stabilizing agents, solubilizing agents. A lozenge core is composed of a solid gel or a lyophilized wafer, containing an phenothiazine in the core. Optionally, the core also contains a stabilizing agent, optionally with one or more additional excipients. Optionally, the upper and lower surfaces of the lozenge core are coated with a chelator, such as sodium EDTA.

In one embodiment, the sublingual composition is a tablet that is a compressed homogenous powder of all of the ingredients. In another embodiment, inactive ingredients, such as the filler and binding agent, and one or more excipients, including the solubilizing agents, are formed into one tablet. The phenothiazine along with filler, binding agent, and other excipients are formed into another tablet. Then the two tablets are placed together and coated to form a single tablet. In some instances, the tablet contains a disintegrant for rapid release of a phenothiazine. Optionally, the tablet and/or the phenothiazine particles are coated with an enteric coating.

In some embodiments, the sublingual composition is a wafer. The wafer is a flat, solid dosage form with a thickness range from 0.1 mm to 1.5 cm or from 0.2 to 5 cm. The wafer contains one or more layers. Each of the layers may be different, or two of the layers, such as the bottom and top layers may have substantially the same composition. In one embodiment, the bottom and top layers surround a core layer containing the phenothiazine. The bottom and top layers may contain one or more excipients, such as a solubilizing agent and a metal chelator. In some instances the outer layers contain disintegrants to allow for rapid release of a phenothiazines.

In some embodiments, a sublingual composition described herein is a capsule. The capsule contains a rapidly dissolving outer shell, which is typically composed of sugars, starches, polymers (and other suitable pharmaceutical materials). The capsule contains powders or granules of agent and excipient. The capsule is designed rapidly release powders or small rapidly dissolving granules into the oral cavity following sublingual administration.

### Methods

Phenothiazines such as, for example, promethazine, prochlorperazine, thioproperazine, fluopromazine, perphenazine, are used in the treatment of nausea and vomiting. Conventionally, phenothiazines are administered via the oral, rectal, intravenous or intramuscular routes. Following intramuscular administration of prochlorperazine edisylate, the drug has an onset of action within ten to twenty minutes and a duration of action of three to four hours. The systemic administration of conventional dosage forms of prochlorperazine is associated with variable blood levels of the drug in patients and side-effects such as drowsiness, dizziness, blurred vision, abnormal menstrual flow, rash, low blood pressure, and/or distorted voluntary movements of the body (dystonic reaction). In some instances, the treatment of nausea or emesis requires administration of multiple doses within a 24 hour period and may exacerbate the side-effects associated with administration of phenothiazines. Provided herein is a method for preventing, reducing, treating, ameliorating or reversing nausea or emesis in a subject comprising intranasally or sublingually administering a therapeutically effective amount of a composition described herein to a subject in need thereof. In some embodiments, the nasal or sublingual compositions described herein are administered after the onset of nausea or emesis. In other embodiments, the nasal or sublingual compositions described herein are administered prophylactically prior to the onset of nausea or emesis.

Intranasal or sublingual administration of any composition described herein allows for rapid absorption of the administered phenothiazine into the circulatory system of a subject and avoids variability in blood levels of the drug that is associated with other modes of administration. Further, the methods described herein provide rapid relief from symptoms of nausea and emesis. The symptoms of nausea or emesis may be associated with any underlying disease or condition, including by way of example, migraines, vertigo, motion sickness, and/or disorders of the inner ear. The symptoms of nausea or emesis may also be a side-effect of any other drug therapy. The intranasal or sublingual dose of phenothiazine (e.g., prochlorperazine) administered to a patient in order to treat nausea or emesis is substantially lower than conventional oral, intramuscular and/or intravenous phenothiazine doses used for the treatment of nausea or emesis while achieving the same therapeutic benefit as a conventional dose of a phenothiazine.

In some instances, a therapeutically effective amount of a composition described herein is prophylactically administered intranasally or sublingually to a subject diagnosed with or suspected of having nausea or emesis associated with a cancer treatment. A cancer treatment is chemotherapy, radiation therapy, antibody therapy, or the like, and/or any combination thereof. Any cancer therapy influences the quality of life of a subject undergoing the cancer therapy. Cancer treatments are routinely associated with emetogenicity that varies based on a subject's gender and age, and/or past medical history, as well as the side effect profile and/or the administration schedule of the cancer treatment(s). Nausea and/or emesis is associated with a variety of cancer therapies and is independent of cancer type or cancer therapy or treatment regimens. Thus the compositions described herein provide relief from symptoms of nausea and/or emesis associated with any cancer therapy associated with any cancer type.

Provided herein is a method for improving the quality of life of a subject undergoing cancer therapy comprising intranasally administering a therapeutically effective amount of a composition described herein to a subject in need thereof and providing rapid relief to the subject suffering from nausea or emesis associated with the cancer therapy. In some instances, the method comprises prophylactic intranasal or sublingual administration of a therapeutically effective amount of a composition described herein to a subject diagnosed with or suspected of having nausea or emesis prior to the start of cancer therapy.

In some embodiments, provided herein is a method for decreasing the dose of a therapeutically effective amount of a phenothiazine necessary for preventing, reducing, treating, ameliorating or reversing nausea or emesis comprising intranasally or sublingually administering a therapeutically effective amount of a composition described herein to a subject in need thereof. In certain instances a decrease in dose is achieved by intranasally or sublingually administering a reduced amount of a phenothiazine compared to the amount of phenothiazine that is administered via an oral, intravenous and/or intramuscular route to achieve a similar therapeutic effect. In some instances, the method comprises prophylactic intranasal or sublingual administration of a reduced amount of a phenothiazine to a subject diagnosed with or suspected of having nausea or emesis. In some instances, a reduced amount of a phenothiazine is prophylactically administered intranasally or sublingually to a subject diagnosed with or suspected of having nausea or emesis associated with a cancer treatment. The reduced dose of phenothiazine ameliorates side effects associated with higher doses of phenothiazine administered via an oral, intravenous and/or intramuscular route while achieving the same anti-emetic effect as a higher dose of phenothiazine.

In some embodiments, provided herein is a reduced side effect method for preventing, reducing, treating, ameliorating or reversing nausea or emesis in a subject in need thereof by intranasally or sublingually administering a therapeutically effective amount of a composition described herein. In certain instances a reduction in side effects is achieved by intranasally or sublingually administering a reduced amount of a phenothiazine compared to the amount of phenothiazine that is administered via an oral, intravenous and/or intramuscular route to achieve a similar therapeutic effect. In some instances, the method comprises prophylactic intranasal or sublingual administration of a therapeutically effective amount of a composition described herein to a subject diagnosed with or suspected of having nausea or emesis. In some instances, a therapeutically effective amount of a composition described herein is administered to a subject diagnosed with or suspected of having nausea or emesis associated with a cancer treatment prior to the start of the cancer treatment and is continued during the cancer treatment. In some embodiments, a pharmaceutical composition described herein is administered at the same time as a cancer treatment regimen. In other embodiments, a pharmaceutical composition described herein is administered sequentially, in any order, with a cancer treatment regimen. By way of example only, a composition described herein is administered prior to the start of a round of chemotherapy and/or radiation, or after completion of a round of chemotherapy and/or radiation therapy, or co-administered during a round of chemotherapy and/or radiation).

In certain embodiments, the dose of phenothiazine (e.g., prochlorperazine) in a composition described herein that is administered to a patient in order to treat nausea or emesis associated with cancer treatment, migraines, vertigo, motion sickness, inner ear disorders or the like is substantially lower than conventional oral, intramuscular and/or intravenous phenothiazine doses used for the treatment of nausea or emesis while achieving the same therapeutic benefit as a conventional dose of a phenothiazine. For example, the current recommended doses of prochlorperazine Oral Dosage Tablets is 5 mg or 10 mg tablet 3 or 4 times daily; Rectal Dose is 25 mg twice daily; Intramuscular dose is initial dose of 5 to 10 mg (1 to 2 mL) injected deeply into the upper outer quadrant of the buttock, with a repeat dose every 3 or 4 hours; Intravenous dose is 2½ to 10 mg (½ to 2 mL) by slow I.V. injection.

In certain embodiments, the dose of phenothiazine (e.g., prochlorperazine) for intransal or sublingual administration of a composition described herein is about 0.1 mg to 5 mg of phenothiazine. In certain embodiments, the dose of phenothiazine (e.g., prochlorperazine) for intranasal or sublingual administration of a composition described herein is 0.1 mg, 0.25 mg, 0.5 mg, 0.75 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, or 5 mg of phenothiazine. In certain embodiments, the dose of phenothiazine for intranasal or sublingual administration of a composition described herein is about 3 mg to 40 mg of phenothiazine. In certain embodiments, the dose of phenothiazine for intranasal or sublingual administration is 0.5 mg, 1 mg, 1.25 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 20 mg, 30 mg, or 40 mg of phenothiazine. In certain embodiments, the dose of phenothiazine for intranasal or sublingual administration is 3 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, or 50 mg of phenothiazine. In certain embodiments, the dose of phenothiazine for intranasal or sublingual administration is less than 2 mg, 5 mg, 7.5 mg, or 10 mg of phenothiazine.

The compositions described herein allow for reduced frequency of dose administration. In some instances, the frequency of administration of a dose of a phenothiazine (e.g., prochlorperazine) composition for intransal or sublingual administration described herein is once a day. In some instances, the frequency of administration of a dose of a phenothiazine (e.g., prochlorperazine) composition for intransal or sublingual administration described herein is twice a day. In some instances, the frequency of administration of a dose of a phenothiazine (e.g., prochlorperazine) composition for intransal or sublingual administration described herein is once every 48 hours, every 36 hours, every 24 hours, every 12 hours, every 10 hours, or every 8 hours.

In some embodiments, a subject in need of a therapeutically effective amount of a composition described herein is diagnosed with or suspected of having migraines, vertigo, motion sickness and/or inner ear disorders. In some embodiments, a subject in need of a therapeutically effective amount of a composition described herein is diagnosed with or suspected of having a cancer including, by way of example, Acute Lymphoblastic Leukemia, Acute Myeloid Leukemia, Adrenocortical Carcinoma, AIDS-Related Cancers, AIDS-Related Lymphoma, Anal Cancer, Appendix Cancer, Astrocytoma, Atypical Teratoid/Rhabdoid Tumor, Basal Cell Carcinoma, Bile Duct Cancer, Bladder Cancer, Bone Cancer, Osteosarcoma and Malignant Fibrous Histiocytoma, Brain Cancer, Bronchial Tumors, Burkitt Lymphoma, Breast Cancer, Cervical Cancer, Chronic Lymphocytic Leukemia, Chronic Myelogenous Leukemia, Colorectal Cancer, Cutaneous T-Cell Lymphoma, Esophageal Cancer, Endometrial Cancer, Eye Cancer, Gallbladder Cancer, Gastric Cancer, Gastrointestinal Stromal Cell Tumor, Head and Neck Cancer, Hodgkin Lymphoma, Hepatocellular Cancer, Pancreatic Cancer, Renal Cell Cancer, Kaposi Sarcoma, Laryngeal Cancer, Liver Cancer, Lung Cancer, Melanoma, Mesothelioma, Multiple Myeloma, Non-Hodgkin Lymphoma, Ovarian Cancer, Oral Cancer, Prostate Cancer, Skin Cancer, Throat Cancer, Thyroid Cancer, Testicular Cancer, Uterine Cancer, Waldenström Macroglobulinemia, or Wilms Tumor.

In some embodiments, a subject in need of a therapeutically effective amount of a composition described herein is a subject undergoing cancer therapy including, by way of example, chemotherapy, radiation therapy, antibody therapy, or the like and/or any combination thereof. In some embodiments, a therapeutically effective amount of a composition described herein is intranasally or sublingually administered to a subject undergoing cancer therapy for reducing, treating, ameliorating and/or reversing nausea or emesis associated with the cancer therapy. In some embodiments, a prophylactically effective amount of a composition described herein is administered intranasally or sublingually to a subject diagnosed with cancer prior to the subject undergoing cancer therapy for preventing, reducing, treating, ameliorating and/or reversing nausea or emesis associated with the cancer therapy.

As used herein, “bioavailability” is the rate and extent to which the active substance, or moiety, which reaches the systemic circulation as an intact drug. The bioavailability of any drug will depend on how well is adsorbed and how much of it escapes being removed from the liver. Bioavailability of a drug can be tested in a variety of ways known in the art.

By way of example, the absolute bioavailability of a drug administered by a formulation described herein is tested by comparing the bioavailability of the test mode of administration against an intravenous reference dose. The bioavailability of an intravenous dose is 100% by definition. For example, in an experiment to determine bioavailability, animals or volunteering humans are given an intravenous injections and corresponding nasal or sublingual doses of a phenothiazine (e.g., prochlorperazine). Urinary or plasma samples are taken over a period of time and levels of the drug over that period of time are determined.

The areas under the curve (AUC), of the plasma drug concentration versus time curves, are plotted for both the intravenous and the nasal or sublingual doses, and calculation of the bioavailability of the formulations is by simple proportion. For example, if the same intravenous and nasal doses are given, and the nasal AUC is 50% of the intravenous AUC, the bioavailability of the nasal formulation is 50%. The absolute bioavailability of any drug is affected by many factors including incomplete absorption, first pass clearance, volume of distribution or a combination of thereof. In addition to the AUC, the peak concentration (or Cₘₐₓ) of the drug concentration in the plasma is also measured and compared to the peak concentration (Cₘₐₓ) of the plasma drug concentration following IV injection of an equivalent concentration the drug. The time to maximal concentration (tₘₐₓ) of the drug in the plasma is also measured.

To determine the relative bioavailability of more than one formulation of a drug (e.g. a formulation with or without a permeation enhancer), bioavailability of the formulations are assessed against each other. For example, a first nasal formulation is assessed against a second nasal formulation. The second formulation is used as a reference to assess the bioavailability of the first. This type of study provides a measure of the relative performance (e.g. absorption and/or bioavailability) of two formulations.

Further, it will be understood that for any method or formulation described herein, the specific dose level and frequency of dosage for any particular subject in need of treatment may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the subject undergoing therapy.

### EXAMPLES

### Example 1

### Nasal Composition

| | % weight by volume |
|---|---|
| Prochlorperazine edisylate | 2 |
| Glycerin | 10 |
| Microcrystalline cellulose | 8 |
| carboxymethylcellulose | 7 |
| sodium | |
| Sodium citrate/citric acid | 10 |
| Polysorbate 80 | 8 |
| Tridecyl maltoside | 2 |
| Benzalkonium chloride | 1.2 |
| Water | Adjust to volume |

Prochlorperazine edisylate is dissolved in water. Microcrystalline cellulose, carboxymethylcellulose sodium are suspended in water, followed by addition of a solution of Polysorbate 80 in water. Tridecyl maltoside is added to the mixture. The solution of Prochlorperazine is added to the suspension. Glycerin, Sodium citrate/citric acid are added to the mixture and the pH is adjusted to between 4.7-5.2. Benzalkonium chloride is added and additional water is added to adjust to volume. The solution is packaged in metered spray bottles.

### Example 2

### Nasal Composition (Preservative Free)

Prochlorperazine hydrochloride is dissolved in sterile water (0.04% solution). Citric acid is added to adjust pH to about 4.5. Cremphor®RH40 (0.02%) is added and the mixture is stirred for 1 hour. The composition is packaged in sterile spray bottles.

### Example 3

### Nasal Composition

| | % weight by volume |
|---|---|
| Prochlorperazine mesylate | 1 |
| Glycerin | 10 |
| Microcrystalline cellulose | 5 |
| Hydroxypropyl cellulose | 8 |
| Sodium citrate/citric acid | 10 |
| sorbitan monooleate | 7 |
| tetradecyl maltoside | 0.5% |
| Benzyl alcohol | 0.9% |
| Water | Adjust to volume |

Prochlorperazine mesylate is dissolved in water. Microcrystalline cellulose, hydroxypropylcellulose are suspended in water, followed by addition of a solution of Polysorbate 80 in water. Tetradecyl maltoside is added to the mixture. The solution of Prochlorperazine is added to the suspension. Glycerin, Sodium citrate/citric acid are added to the mixture and the pH is adjusted to between 5.0-6.5. Benzyl alcohol is added and additional water is added to adjust to volume. The solution is packaged in metered spray bottles.

### Examples 4-7

Formulations comprising promethazine, fluopromazine, thioridazine and acetophenazine are prepared according to procedure in Example 3.

### Example 8

### Nasal Composition

| | % weight by volume |
|---|---|
| Prochlorperazine | 0.5 |
| hydrochloride | |
| Glycerin | 10 |
| Carbopol 934 | 4 |
| carboxymethylcellulose | 10 |
| sodium | |
| Phosphate buffer | ~10 |
| Polysorbate 80 | 5 |
| dodecyl maltoside | 6% |
| Methyl paraben | 0.7% |
| Water | Adjust to volume |

Prochlorperazine hydrochloride is dissolved in water. Carbopol 934, carboxymethylcellulose sodium are suspended in water, followed by addition of a solution of Polysorbate 80 in water. Dodecyl maltoside is added to the mixture. The solution of Prochlorperazine is added to the suspension. Glycerin, phosphate buffer are added to the mixture and the pH is adjusted to between 5.5-7.1. Methyl paraben is added and additional water is added to adjust to volume. The solution is packaged in metered spray bottles.

### Example 9

### Nasal Composition, without Preservative

| | % weight by volume |
|---|---|
| Prochlorperazine maleate | 0.1 |
| Propylene glycol | 10 |
| ethanol | 5 |
| carboxymethylcellulose | 10 |
| sodium | |
| Sodium citrate/citric acid | 10 |
| sorbitan monolaurate | 8 |
| dodecyl sucrose | 0.1 |
| Water | Adjust to volume |

Prochlorperazine maleate is dissolved in water. Carboxymethylcellulose sodium is suspended in water, followed by addition of a solution of Polysorbate 80 in water. Dodecyl sucrose is added to the mixture. The solution of Prochlorperazine is added to the suspension. Propylene glycol, Sodium citrate/citric acid are added to the mixture and the pH is adjusted to between 4.7-5.2. The solution is packaged in metered spray bottles.

### Example 10

### Nasal Composition, Suspension

| | % weight by volume |
|---|---|
| Prochlorperazine | 0.1 |
| carboxymethylcellulose | 10 |
| sodium | |
| Phosphate buffer | 10 |
| Poloxamer 407 | 5 |
| dodecyl sucrose | 0.1 |
| Water | Adjust to volume |

Micronized prochlorperazine is suspended in water. Carboxymethylcellulose sodium is suspended in water, followed by addition of a solution of poloxamer in water. Dodecyl sucrose is added to the mixture. The suspension of Prochlorperazine is added to the mixture. Phosphate buffer is added to the mixture and the pH is adjusted to between 7.0-7.8. The solution is packaged in metered spray bottles.

### Example 11

### Sublingual Composition, Effervescent

| | % weight |
|---|---|
| Prochlorperazine maleate | 15 |
| lactose | 10 |
| mannitol | 7 |
| cornstarch | 8.5 |
| Povidone K30 | 15 |
| Magnesium stearate | 30.5 |
| Acesulfame K | 4 |
| Citric acid | 5 |
| Sodium carbonate | 5 |

Prochlorperazine maleate is dissolved in 6 ml of anhydrous ethanol and is added to Povidone K30 contained in a blender bowl while being mixed at 40-50 rpm. Mixing is continued for 15 minutes at 40 rpm. Mixture is passed through a #30 (U.S. Standard) screen and dried. The corn starch is added by passing it through a #30 (U.S. Standard) screen. The powder mix is mixed in a blender for 35 minutes at 40 rpm. Magnesium stearate is added by passing it through a #60 (U.S. Standard) screen and mixing is continued for an additional 3 minutes at 40 rpm. Citric acid, sodium carbonate and acesulfame are added and mixing is continued for an additional 5 minutes at 40 rpm. The lubricated mixture is compressed using an 8/32′ hexagonal tablet tool.

### Example 12

### Sublingual Composition, Rapid Release

91.0 g of mannitol and 1.0 g of sodium lauryl sulfate and 500 mg of micronized prochlorperazine are mixed in a V-mixer over a period of 24 hours. 2.0 g of Ac-Di-Sol (disintegrant) is admixed for an additional 2 hours. Finally, 0.5 g of magnesium stearate is admixed for 2 minutes. The resulting tablet mass is compacted into tablets.

### Example 13

### Animal Model to Test Efficacy of a Nasal Formulation

In this model animals are divided into two groups. One group of animals are challenged with cisplatin (10 mg/kg ip). 4 hour after cisplatin dose, nasal formulations comprising phenothiazens are administered to the animals. The second group of animals are challenged with cisplatin (10 mg/kg ip). 4 hour after cisplatin dose, oral syrup formulations comprising phenothiazens are administered to the animals. The anti-emetic response in each group is quantified via direct observation of retching and vomit frequency. A reduction in emesis in the first group compared to the second group indicates efficacy of nasal formulations that deliver phenothiazines directly into the circulatory system over formulations that require absorption via the gastrointestinal tract.

### Example 14

### Clinical Trial

This is a Phase II trial to determine efficacy of a nasal formulation of prochlorperazine in preventing nausea and vomiting in patients undergoing chemotherapy for cancer.

This is an interventional study. The study will be carried out with supportive care. The study is a randomize double-blinded study with placebo control.

A cohort of 200 cancer patients will be divided into two groups, one receiving prochlorperazine and the other group receiving placebo. The prochlorperazine nasal composition (each metered dose administers 2 mg) will be administered one day prior to start of a chemotherapy cycle for the patient and once daily administration will be continued until patient displays no significant emesis or nausea.

### Inclusion Criteria:

Histologically or cytologically confirmed solid tumors. Receiving a moderately emetogenic chemotherapy regimen for the first time. Scheduled to receive cyclophosphamide ≦1,500 mg/m̂2 IV and/or doxorubicin hydrochloride ≧40 mg/m̂2 IV given as single doses on day 1 of chemotherapy regimen. Patients on combination regimens with these agents are also eligible. No concurrent moderately emetogenic chemotherapy (Hesketh Level 3-4) after day 1 of the study period. Hesketh Level 1-2 chemotherapy on days 2-5 allowed. No other physical causes for nausea or vomiting not related to chemotherapy administration (i.e., bowel obstruction). No recent history of unexplained nausea or vomiting or history of frequent nausea or vomiting. No uncontrolled primary or metastatic CNS tumor (including those with uncontrolled seizures).

### Primary Outcome Measures:

Total protection (i.e., no vomiting, no rescue therapy, and no nausea as indicated by responses to the Daily Assessment of Nausea and Vomiting questionnaire during the overall [0-120 hour] period)

### Secondary Outcome Measures:

No vomiting, no significant nausea, and no nausea evaluated for the acute (0-24 hour), delayed (24-120 hour), and overall (0-120 hour) periods; Complete protection and complete response for the acute, delayed, and overall periods

Patients complete a Daily Assessment of Nausea and Vomiting questionnaire before and after the administration of chemotherapy. Patients are followed to the completion of a full cycle of chemotherapy (days 14-28).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A pharmaceutical composition comprising a phenothiazine or phenothiazine derivative, or a salt thereof, or any combination thereof, and at least one permeation enhancer, wherein the pharmaceutical composition is formulated for nasal administration.

2. The composition of claim 1 wherein the phenothiazine is prochlorperazine or a prochlorperazine derivative, or a salt thereof, or any combination thereof.

3. The composition of claim 1, wherein the permeation enhancer reduces the concentration of the phenothiazine in the composition necessary to exert a therapeutic effect in a subject compared to a composition without the permeation enhancer.

4. The composition of claim 1, further comprising an alkyl glycoside having a hydrophobic alkyl group joined by a linkage to a hydrophilic saccharide.

5. The composition of claim 1, wherein the at least one permeation enhancer is selected from:
(a) a surfactant;
(b) a bile salt;
(c) a phospholipid additive,
(d) a mixed micelle
(e) a liposome;
(f) an alcohol;
(g) an enamine;
(h) an NO donor compound;
(i) a long-chain amphipathic molecule;
(j) a small hydrophobic penetration enhancer;
(k) sodium or a salicylic acid derivative;
(l) a glycerol ester of acetoacetic acid;
(m) a cyclodextrin or beta-cyclodextrin derivative;
(n) a medium-chain fatty acid;
(o) a chelating agent;
(p) an amino acid or salt thereof;
(q) an N-acetylamino acid or salt thereof;
(r) an enzyme degradative to a selected membrane component; and
(s) an alkyl glycoside.

6. The composition of claim 5, wherein the alkyl glycoside is selected from dodecyl maltoside, tridecyl maltoside, tetradecyl maltoside, sucrose mono-dodecanoate, sucrose mono-tridecanoate, and sucrose mono-tetradecanoate.

7. The composition of claim 1, further comprising a delivery enhancing agent.

8. The composition of claim 7, wherein the delivery enhancing agent is selected from a charge-modifying agent, a pH control agent, a degradative enzyme inhibitory agent, a mucolytic or mucus clearing agent, a chitosan, and a ciliostatic agent.

9. The composition of claim 1, wherein the pH of the composition is between about 5 and about 8.

10. A method of treating nausea or emesis in a subject comprising intranasally administering the composition of claim 1 to a subject in need thereof.

11. The method of claim 10, wherein the subject is undergoing treatment for cancer.

12. A method of making a pharmaceutical composition for nasal delivery comprising formulating a pharmaceutical composition comprising at least one phenothiazine or phenothiazine derivative and at least one permeation enhancer, wherein said pharmaceutical composition is suitable for nasal administration.
